(19) United States
(12) Patent Application Publication
(10) Pub. No.: US 2014/0250556 A1
(43) Pub. Date: Sep. 11, 2014

(54) EYE PATCH

(71) Applicant: Musarat Eliza Awan, Bramcote (GB)

(72) Inventor: Musarat Eliza Awan, Bramcote (GB)

(21) Appl. No.: 14/234,911

(22) PCT Filed: Jul. 24, 2012
(86) PCT No.: PCT/GB2012/051768

§ 371 Date: May 27, 2014

(30) Foreign Application Priority Data
Jul. 26, 2011 (GB) 1112778.4

(51) Int. Cl. A61F 9/04

(52) U.S. Cl. CPC A61F 9/045
USPC 2/15; 66/171

(57) ABSTRACT

The invention provides an eye patch (2). The eye patch comprises a concave section (4), which, in use, is adapted to cover a subject's eye. The eye patch comprises a peripheral edge (6) extending around at least a portion of the concave section and comprising an adhesive (12), which, in use, is adapted to secure the eye patch to the subject.

The present invention relates to eye patches, and to uses thereof for treating eye disorders, such as amblyopia, conjunctivitis, and post-operative eye management, such as lid, squint, corneal surgery or ocular trauma.

Amblyopia, otherwise known as lazy eye, is a disorder of the visual system that is characterized by poor vision or lack of vision in an eye that is otherwise physically normal. The problematic eye is typically healthy but, due to development issues, the visual information received at the back of the eye and the neural information interpreted by the brain is not well transmitted or recognized. This results in persistent blurred vision that continues irrespective of wearing corrective glasses or a contact lens(s). It usually occurs in one eye, rarely in both eyes. Amblyopia affects approximately 2% to 5% of children, although it frequently affects 2%-4.4% of the childhood population.

Amblyopia can be treated using an occlusive eye patch. Treatment duration occurs in the first decade of life, after which usually no further development continues as the brain's visual systems becomes less plastic or resilient to responses received from treatment. Persistent loss of vision reduces the person's ability to perform some or all of the visual tasks that can affect employment prospects. The procedure, to date, is to cover the better-seeing eye with a patch in order to stimulate the eye with poorer vision. Studies have demonstrated that this technique, known as patching, is effective. The use of an occlusive eye patch has been the most widely and preferred form of first line of standard conventional treatment for treating amblyopia, which has therefore established itself as a routine part of clinical care. Accordingly, the use of the eye patch is likely to remain the accepted method of treatment amongst clinicians, consultants and children, and likely to continue for many years to come.

However, the use of patching treatment fails in many cases mainly due to the lack of co-operation from the child to undertake the continual treatment needed. Other problems associated with patching include victimisation, poor cosmetic appearance of the patch, discomfort during treatment and a lack of confidence caused through treatment. Results have also shown that the duration of treatment is long, expensive, with poor outcomes, and may not reach the accepted level of vision at discharge. Hence, the resultant outcome of ineffective treatment is residually amblyopic children who remain residual amblyopic adults with a reduced quality of life and who may be significantly affected with age-related eye diseases, particularly involving visual tasks that affects employment prospects, such as the Armed Forces.

There is therefore a need to provide improved devices and methods for use in the treatment of a wide range of eye diseases, such as amblyopia.

Thus, according to a first aspect of the present invention, there is provided an eye patch comprising a concave section, which, in use, is adapted to cover a subject's eye, and a peripheral edge extending around at least a portion of the concave section and comprising an adhesive, which, in use, is adapted to secure the eye patch to the subject.

Advantageously, the concave section of the eye patch allows the subject to move his or her eye and/or blink the eye covered by the patch in comfort and, importantly, without obstruction. Furthermore, the self-adhesive nature of the peripheral edge means that the subject can wear the patch without the need for elastic or string, or the like. Accordingly, the eye patch is cosmetically acceptable, effective and comfortable for the subject, especially children, thereby reducing the negative impact, which would upset family dynamics during treatment. Current eye patches are ineffective due to negative issues surrounding comfort and perception. However, the ergonomical conceptual design of the eye patch of the invention significantly improves its wearability whilst appealing to children as an accessory rather than as a medical device. In addition, it is believed that the eye patch of the invention will achieve better visual outcomes than known patches, thus shortening treatment duration and reducing the unnecessary expenditure incurred on the healthcare sector for treating the disease.

The eye patch, and preferably the concave section thereof, may be adapted, in use, to prevent light transmittance into the eye during treatment. The eye patch, and preferably the concave section thereof, may comprise an opaque material. Thus, the eye patch may be described as being an occlusive eye patch. The curvature of the concave section may be sufficient such that, in use, as the subject blinks, his or her eyelid (or eye lashes) does not contact, or reduces contact with, any part of the patch, including the concave section. In addition, the eye patch is adapted, in use, to retain skin moisture and hydration, and allow air (and heat) to circulate underneath the patch.

The average diameter of the eye patch may be between about 3 cm and about 8 cm, or between about 3 cm and 8.5 cm, or between about 4 cm and about 7 cm, or between about 4 cm and 7.5 cm, or between about 5 cm and about 6 cm, or between about 5 cm and 6.5 cm. The eye patch may be between about 0.1 mm and 3 mm think, or between about 0.5 mm and 2 mm thick, or between about 0.8 mm and 1.7 mm thick.

The eye patch may be contoured. Thus, the eye patch may comprise a smooth rounded apex, as seen from the exterior of the patch and/or a smooth concaved cone, as seen from the interior of the patch. The eye patch may comprise an ergonomically contoured design, which is capable of permitting free movement of the eye whilst being worn by the subject.

The eye patch may comprise a non-toxic material. The inventor has found that various different materials may be used for the eye patch, and in particular, the concave section. In one embodiment, the eye patch may comprise a knitted or woven material. For example, suitable knitted or woven materials may include cotton, wool, silk, linen, cashmere, nylon, rayon, satin, viscose, hemp, acetate, acrylic, or polyester etc., or any combinations of these materials. In another embodiment, the eye patch may comprise a non-woven material, which may be a single-line weft or warp knitted non-woven material. Examples of such non-woven materials may include polyamide, felt, cerex, and non-woven or spun-bound polypropylene with glue intact. In another embodiment, the eye patch may comprise silicone/polyurethane/elastomers or high-tech polymer medical grades or coatings of different non-toxic materials that are shape compatible.

For example, polyamide and/or polyester have been found to be particularly useful, because such materials are durable, capable of transporting and absorbing moisture, breathable due to increased airflow, flame retardant, and opaque. The patch may comprise two layers of fabric joined together by microfilaments. Such a material is known as a “spacer fabric”, which may be either woven or knitted spacer fabric. The eye patch may comprise an antimicrobial agent, for example an antibacterial agent. The patch may be coated or impregnated with the antimicrobial agent.

The eye patch may comprise a fabric which is dyed, patterned or printed. For example the print may be of a children's character, such as a Disney-based character, or a CBBC-based character, or own illustrative designed characters. The eye patch may be suitable for both genders. The eye patch may be dyed, printed, knitted, imprinted or moulded

The peripheral edge may surround the entire concave section, which may therefore be described as being an inner section. The peripheral edge may be adapted, in use, to conform to the shape of the subject's eye socket (i.e. orbit) for facilitating adhesion to the skin on the socket. The peripheral edge may be substantially planar. The average width of the peripheral edge may be between about 1 mm and 10 mm, or between about 2 mm 8 mm, or between about 3 mm and 6 mm.

The angle of the junction where the concave section and peripheral edge meet may be between about 110° and 175°, or between about 125° and 165°, or between about 140° and 150°. The inventor has found that such an angle provides optimum contact between the adhesive peripheral edge and the eye socket, while still leaving sufficient room between the concave section and the subject's eye and eye lid.

The peripheral edge may comprise an adhesive polymer layer or hot melt adhesive or polyurethane and/or combination of hydro-selective adhesive. For example, the adhesive may comprise silicone resin. Advantageously, silicone resin has been found to minimise skin trauma and allergies commonly associated with glue-based self-adhesives currently used in known eye patches. The eye patch, preferably the adhesive region thereof, may comprise Aloe vera properties to minimise skin hydration known to occur through constant wear of the patch. The adhesive may comprise a silicone/polymer layer with Aloe vera properties. This layer ensures optimal performance of the eye patch, such that it remains comfortable throughout wearing time.

Advantageously, the patch of the invention is eco-friendly and can be washed, minimising unnecessary wastage. The eye patch may be used for the treatment of disorders of the eye.

Thus, according to a second aspect, there is provided an eye patch according to the first aspect, for use in therapy.

According to a third aspect, there is provided an eye patch according to the first aspect, for use in treating an eye disorder.

For example, the eye disorder may be amblyopia, conjunctivitis, post-operative management such as lid, squint, corneal surgery or ocular trauma. The patch may be used for treating amblyopia. The patch may be capable of blocking out partial light, primarily aimed at treating mild amblyopia.

According to a fourth aspect, there is provided a method of treating an eye disorder in a subject, the method comprising attaching, to a subject in need of such treatment, an eye patch according to the first aspect.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:-

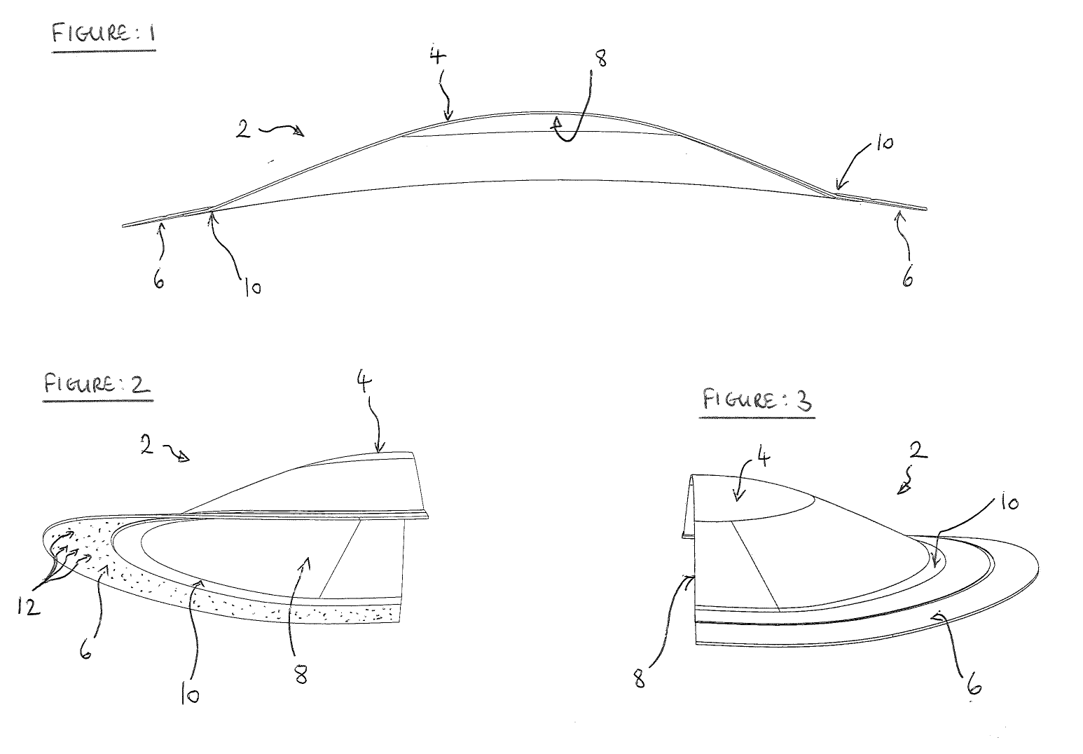

FIG. 1 is a schematic side view of on embodiment of an eye patch of the invention;

FIG. 2 is an enlarged perspective view from below of the eye patch shown in FIG. 1; and

FIG. 3 is an enlarged perspective view from above of the eye patch shown in FIG. 1.

### EXAMPLE

Referring to FIGS. 1-3, there is shown an embodiment of an occlusive eye patch 2 according to the invention, which is called the OvTEP occlusive eye patch 2. The patch 2 is regarded as being a class I medical device, and can be worn by a subject, such as a child, particularly for the treatment of amblyopia or other eye disorders, such as conjunctivitis.

The eye patch 2 is substantially spherical in plan view, but has a concave contour when viewed from below, as shown in the Figures. The concave nature of the patch 2 provides room for the subject's eye to move freely, and the eye lid to open and close unhindered when blinking. The patch 2 is, therefore, very comfortable to wear, such that the subject is happy to wear it for extended periods of time, thereby improving the treatment regime. The eye patch 2 is opaque and so does not allow any light to permeate therethrough at any point. It is provided in two sizes, i.e. a Junior size for treating children up to four years of age, and a Regular size for treating children who are four years old and over. Examples of the dimensions of the eye patch 2 are 5.5 cm and 7.8 cm in length, 5.2 cm and 6.5 cm in width or 3.8 cm and 5 cm in width, and 0.1 mm and 1.7 mm and 0.5 mm and 2 mm thick.

The patch 2 has an inner section 4, which, in use, covers the subject's eye, and an outer peripheral edge 6, by which the patch 2 is adhered to the user. The inner section 4 and edge 6 meet at junction 10 forming an angle of about 120° to about 135°. This angle reflects the edge of the orbital bone area around the eye and the beginning of the contoured shape of the eye patch 2 heading towards its smooth apex. The patch 2 is lightweight and mouldable, the inner section 4 being made of a breathable material, for example a polyamide/polyester spacer composite material (e.g. weft-knitted 3D-spacer technology). The material consists of two layers of a composite of polyamide and polyester held together by microfilaments, and is about 1.5 mm, 1.7 mm or 2.0 mm in thickness. The peripheral edge 6 is made of a single layer of the same material as the inner section 4.

The inner section 4 is coated in an antimicrobial agent, in order to prevent eye infections. Referring to FIG. 2, a lower surface of the peripheral edge 6 includes an adhesive material 12, by which the patch 2 is adhered to the subject's skin. The adhesive material 12 can be a layer of glue or silicone resin, or the like, and may have additional Aloe Vera properties, which minimises skin hydration known to occur through constant wear of the patch.

In use, the subject carefully positions the eye patch 2 over the eye, with the inner section 4 positioned directly over the eye/eye lid, and the peripheral edge 6 positioned around the eye/eye lid. The subject ensures that it is still possible to move the eye covered by the patch 2 and blink normally without the eye lid contacting the concave interior surface 8 of the inner section 4 of the patch 2. Enough room is provided underneath the patch 2 so that even the subject's eyelashes do not come into contact with surface 8. The adhesive nature of the adhesive layer 12 on the peripheral edge 6 is such that it can last for several hours when applied to the subject. The contoured exterior of the eye patch 2 is sufficiently robust to maintain its shape, despite physical trauma even when it is worn.

Advantages of the eye patch 2 reside in the fact that it is concave in shape, thereby allowing a subject wearing the patch 2 to blink freely and move the eye without any obstruction. In addition, the eye patch 2 comprises the adhesive peripheral edge 6 which ensures that the patch 2 remains adhered over a subject's eye avoiding the need for elastic or string or the like. Furthermore, the patch 2 is made of a breathable material such that the moisture and air flow can reach the eye, thereby keeping the temperature constant. Thus, the patch 2 is breathable with pressure sensitive skin contact with Aloe vera properties that may be inbuilt into the patch 2, sterile and anti-bacterial, hypoallergenic, secretion absorption, flame retardant and durable. Due to the type of fabric used in the patch 2, it will not breakdown or decompose during the duration of treatment. Finally, the glue-based silicone/polymer resin will have time-release benefits ensuring that full treatment is exercised despite the physical removal of the patch from the skin.

1. An eye patch comprising a concave section, which, in use, is adapted to cover a subject's eye, and a peripheral edge extending around at least a portion of the concave section and comprising an adhesive, which, in use, is adapted to secure the eye patch to the subject.

2. An eye patch according to claim 1, wherein the eye patch is adapted, in use, to prevent light transmittance into the eye during treatment.

3. An eye patch according to claim 1, wherein the eye patch comprises an opaque material.

4. An eye patch according to claim 1, wherein the curvature of the concave section is sufficient such that, in use, as the subject blinks, his or her eyelid (or eye lashes) reduces contact with any part of the patch.

5. An eye patch according to claim 1, wherein the eye patch comprises a smooth rounded apex, as seen from the exterior of the patch and/or a smooth concaved cone, as seen from the interior of the patch.

6. An eye patch according to claim 1, wherein the eye patch comprises a knitted or woven material, for example cotton, wool, silk, linen, cashmere, nylon, rayon, satin, viscose, hemp, acetate, acrylic, or polyester etc., or any combinations of these materials.

7. An eye patch according to claim 1, wherein the eye patch comprises a non-woven material, for example a single-line weft or warp knitted non-woven material, such as polyamide, felt, cerex, and non-woven or spun-bound polypropylene with glue intact, polyurethane, silicone or combination of materials.

8. An eye patch according to claim 1 wherein the eye patch comprises polyamide and/or polyester.

9. An eye patch according to claim 1, wherein the patch comprises two layers of fabric joined together by microfilaments.

10. An eye patch according to claim 1, wherein the eye patch comprises an antimicrobial agent, for example an antibacterial agent.

11. An eye patch according to claim 1, wherein the eye patch comprises a fabric which is dyed, patterned or printed.

12. An eye patch according to claim 1, wherein the peripheral edge surrounds the entire concave section.

13. An eye patch according to claim 1, wherein the peripheral edge is adapted, in use, to conform to the shape of the subject's eye socket for facilitating adhesion to the skin on the socket.

14. An eye patch according to claim 1, wherein the peripheral edge is substantially planar.

15. An eye patch according to claim 1, wherein the angle of the junction where the concave section and peripheral edge meet is between about 110° and 175°, or between about 125° and 165°, or between about 140° and 150°.

16. An eye patch according to claim 1, wherein the peripheral edge comprises an adhesive polymer layer or hot melt adhesive or polyurethane and/or combination of hydro-selective adhesive.

17. An eye patch according to claim 1, wherein the adhesive comprises silicone resin.

18. An eye patch according to claim 1 wherein the adhesive comprises a silicone/polymer layer with Aloe Vera properties.

19. An eye patch according to claim 1, for use in therapy.

20. An eye patch according to claim 1, for use in treating an eye disorder.

21. A eye patch according to claim 20, wherein the eye disorder is amblyopia, conjunctivitis, post-operative management such as lid, squint, corneal surgery or ocular trauma.

22. A method of treating an eye disorder in a subject, the method comprising attaching, to a subject in need of such treatment, an eye patch according to claim 1.
